(19) United States
(12) Patent Application Publication
(10) Pub. No.: US 2024/0331831 A1
(43) Pub. Date: Oct. 3, 2024

(54) INFORMATION PROCESSING DEVICE, INFORMATION PROCESSING METHOD, PROGRAM RECORDING MEDIUM, AND MODEL BEING TRAINED

(71) Applicant: SEIKO EPSON CORPORATION, Tokyo (JP)

(72) Inventor: Ienari OBINATA, Matsumoto-shi (JP)

(73) Assignee: SEIKO EPSON CORPORATION, Tokyo (JP)

(21) Appl. No.: 18/618,126

(22) Filed: Mar. 27, 2024

(30) Foreign Application Priority Data
Mar. 28, 2023 (JP) 2023-050816

(51) Int. Cl. G16H 20/30; G06N 20/00

(52) U.S. Cl. CPC G16H 20/30; G06N 20/00

(57) ABSTRACT

An information processing device includes a target person information acquisition unit configured to acquire target person information about a target person, an exercise evaluation information acquisition unit configured to acquire exercise evaluation information indicating evaluation of an exercise of the target person, a storage unit configured to store a trained model that outputs instruction content in which, when the target person information and the exercise evaluation information are input, evaluation of exercise information of the target person is estimated to be improved, and a report output unit configured to input the target person information and the exercise evaluation information to the trained model and output an instruction report including the instruction content output from the trained model.

The present application is based on, and claims priority from JP Application Serial Number 2023-050816, filed Mar. 28, 2023, the disclosure of which is hereby incorporated by reference herein in its entirety.

### BACKGROUND

### 1. Technical Field

The present disclosure relates to an information processing device, an information processing method, a program recording medium, and a model being trained.

### 2. Related Art

In an exercise analysis device described in JP-A-2022-61784, a difference in a physical exercise of a target user with respect to a target physical exercise to be aimed at for the physical exercise of the target user is extracted based on exercise data of the physical exercise of the target user and result data generated by the physical exercise, and advice for reducing the difference is presented to the target user (see JP-A-2022-61784).

However, in the related art, since information for each target person such as the age or the body shape of the target person is not acquired, it may be difficult to give an instruction suitable for each target.

### SUMMARY

In order to solve the above problems, one aspect is an information processing device including a target person information acquisition unit configured to acquire target person information about a target person, an exercise evaluation information acquisition unit configured to acquire exercise evaluation information indicating evaluation of an exercise of the target person, a storage unit configured to store a trained model that outputs instruction content in which, when the target person information and the exercise evaluation information are input, evaluation of exercise information of the target person is estimated to be improved, and a report output unit configured to input the target person information and the exercise evaluation information to the trained model and output an instruction report including the instruction content output from the trained model.

In order to solve the above problems, one aspect is an information processing method including acquiring target person information about a target person using a target person information acquisition unit of an information processing device, acquiring exercise evaluation information indicating evaluation of an exercise of the target person using an exercise evaluation information acquisition unit of the information processing device, storing, using a storage unit of the information processing device, a trained model that outputs instruction content in which, when the target person information and the exercise evaluation information are input, evaluation of exercise information of the target person is estimated to be improved, and inputting the target person information and the exercise evaluation information to the trained model and outputting an instruction report including the instruction content output from the trained model using a report output unit of the information processing device.

In order to solve the above problems, one aspect is a program recording medium configured to record a program, the program causing a computer to execute a target person information acquisition function configured to acquire target person information about a target person, an exercise evaluation information acquisition function configured to acquire exercise evaluation information indicating evaluation of an exercise of the target person, and a report output function configured to input the target person information and the exercise evaluation information to a trained model stored in a storage unit configured to store the trained model that outputs instruction content in which, when the target person information and the exercise evaluation information are input, evaluation of exercise information of the target person is estimated to be improved, and output an instruction report including the instruction content output from the trained model.

In order to solve the above problems, one aspect is an information processing device including an instruction content acquisition unit configured to acquire instruction content based on target person information including a plurality of parameters related to a target person and exercise evaluation information about evaluation of an exercise of the target person, an exercise information evaluation unit configured to acquire the exercise evaluation information about the evaluation of the exercise of the target person after the instruction, and a storage unit configured to store a model being trained in which, when the instruction content and the exercise evaluation information after the instruction are input, an effective parameter among the plurality of parameters of the target person information is analyzed.

In order to solve the problems, one aspect is a model being trained in which, when instruction content based on target person information including a plurality of parameters related to a target person and exercise evaluation information about evaluation of an exercise of the target person, and the exercise evaluation information after the instruction are input, an effective parameter among the plurality of parameters of the target person information is analyzed.

In order to solve the above problems, one aspect is an information processing method including acquiring instruction content based on target person information including a plurality of parameters related to a target person and exercise evaluation information about evaluation of an exercise of the target person using an instruction content acquisition unit of an information processing device, acquiring the exercise evaluation information about the evaluation of the exercise of the target person after the instruction using an exercise information evaluation unit of the information processing device, and storing, using a storage unit of the information processing device, a model being trained in which, when the instruction content and the exercise evaluation information after the instruction are input, an effective parameter among the plurality of parameters of the target person information is analyzed.

### BRIEF DESCRIPTION OF THE DRAWINGS

FIG. 1 is a diagram showing a schematic configuration example of an information processing device according to an embodiment.

FIG. 2 is a diagram showing an example of input and output of a trained model according to the embodiment.

FIG. 3 is a diagram showing a first table representing an example of target person information according to the embodiment.

FIG. 4 is a diagram showing a second table representing an example of correspondence between a BMI value and a determination result according to the embodiment.

FIG. 5 is a diagram showing a third table representing an example of correspondence between a Rohrer index and a determination result according to the embodiment.

FIG. 6 is a diagram showing an example of a series of movements for kicking a ball according to the embodiment.

FIG. 7 is a diagram showing exercise information according to the embodiment and showing an example of a relationship between a time and a speed at the time of swinging a leg or the like.

FIG. 8 is a diagram showing exercise information of a plurality of patterns according to the embodiment and showing an example of a relationship between a time and a speed at the time of swinging a leg or the like.

FIG. 9 is a diagram showing a schematic example of a first instruction report according to the embodiment.

FIG. 10 is a diagram showing a fourth table representing an example of first target person information according to the embodiment.

FIG. 11 is a diagram showing an example of a relationship between a time and a speed in exercise information of the first target information according to the embodiment.

FIG. 12 is a diagram showing a fifth table representing an example of second target person information according to the embodiment.

FIG. 13 is a diagram showing an example of a relationship between a time and a speed in exercise information of a second target person according to the embodiment.

FIG. 14 is a diagram showing a sixth table representing an example of third target person information according to the embodiment.

FIG. 15 is a diagram showing an example of a relationship between a time and a speed in exercise information of a third target person according to the embodiment.

FIG. 16 is a diagram schematically showing an example of generation of teacher data according to the embodiment.

FIG. 17 is a diagram schematically showing an example of analysis using a model being trained according to the embodiment.

FIG. 18 is a diagram schematically showing an example of analysis content using a model being trained according to the embodiment.

### DESCRIPTION OF EMBODIMENTS

Embodiments will be described with reference to the drawings.

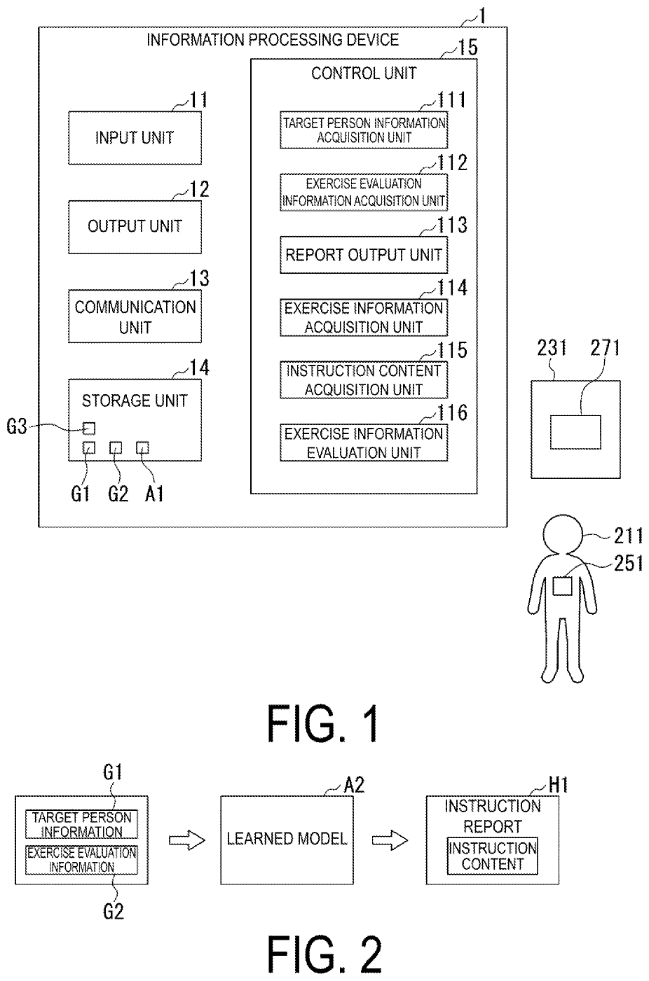

FIG. 1 is a diagram showing a schematic configuration example of an information processing device 1 according to the embodiment.

The information processing device 1 includes an input unit 11, an output unit 12, a communication unit 13, a storage unit 14, and a control unit 15.

The control unit 15 includes a target person information acquisition unit 111, an exercise evaluation information acquisition unit 112, a report output unit 113, an exercise information acquisition unit 114, an instruction content acquisition unit 115, and an exercise information evaluation unit 116.

In addition, FIG. 1 shows a target person 211, a first detection device 251 attached to the target person 211, a tool 231, and a second detection device 271 attached to the tool 231. The target person 211 is a person who receives instruction of an exercise.

Also, although FIG. 1 shows one target person 211, a plurality of target persons are present in this embodiment. The tool 231 is a tool used for the exercise performed by the target person 211.

The first detection device 251 and the second detection device 271 are devices that detect information for evaluating the exercise performed by the target person 211. The information may be information of any physical quantity and may be, for example, acceleration, position information, angular velocity, or the like.

In specific examples, various exercises such as soccer, baseball, basketball, golf, marathon, and the like may be adopted as the exercise.

In addition, examples of the tool may include, for example, a ball in soccer, a bat or a ball in baseball, a ball in basketball, a club in golf, and the like.

Further, for example, any one of the first detection device 251 and the second detection device 271 may be used, or both may be used.

Also, each of the first detection device 251 and the second detection device 271 may be configured using, for example, sensors that detect desired information.

The information detected by the first detection device 251 and the second detection device 271 may be called data, detected values, or the like.

The input unit 11 performs, for example, processing of inputting information in accordance with a user's operation and processing of inputting information from another device. The output unit 12 performs, for example, processing of outputting information to another device. The other device may be a display device, and in this case, the information output from the output unit 12 is displayed on a screen of the display device. As another example, the other device may be a printing device, and in this case, the information output from the output unit 12 is printed out by the printing device. The communication unit 13 communicates with other devices. The communication unit 13 is configured to communicate with, for example, the first detection device 251 and the second detection device 271. In addition, the communication unit 13 may be configured to communicate with other devices (not shown) such as a printing device.

Also, in this embodiment, the communication unit 13 is shown separately from the input unit 11 and the output unit 12, but the function of the communication unit 13 may be included in the functions of the input unit 11 and the output unit 12, for example.

The storage unit 14 stores information.

In this embodiment, the storage unit 14 stores a learning model A1, target person information G1, exercise evaluation information G2, and exercise information G3.

The learning model A1 is a model for machine learning and may be, for example, a pre-learning state, an on-learning state, and a learned state.

The target person information G1 is information about the target person 211.

The exercise evaluation information G2 is information indicating evaluation of an exercise of the target person 211.

The exercise information G3 is information about the exercise of the target person 211.

Also, two or more of the target person information G1, the exercise evaluation information G2, and the exercise information G3 may be associated with each target person 211. The association may be called correspondence or the like.

Here, for example, the information processing device 1 may acquire the exercise evaluation information G2 of the target person 211 from the outside, or may acquire the exercise information G3 of the target person 211 from the outside and generate the exercise evaluation information G2 based on the acquired exercise information G3.

Also, in the example shown in FIG. 1, a state in which the learning model A1, the target person information G1, the exercise evaluation information G2, and the exercise information G3 are stored in the storage unit 14 is shown, but there may be a state in which some or all of these are not stored in the storage unit 14 depending on a timing of processing or the like.

The control unit 15 performs various types of processing and control.

In this embodiment, the information processing device 1 is configured using a computer.

The control unit 15 includes a processor such as a central processing unit (CPU) and performs various types of processing and control by executing a predetermined program using the processor.

The program may be stored in the storage unit 14, for example.

The target person information acquisition unit 111 acquires the target person information G1 via, for example, the input unit 11, the communication unit 13, or the storage unit 14.

The exercise evaluation information acquisition unit 112 acquires the exercise evaluation information G2 via, for example, the input unit 11, the communication unit 13, or the storage unit 14.

The report output unit 113 generates an instruction report H1 and outputs the instruction report H1. This output may be, for example, a display output or a paper output from a printing device.

Here, the instruction report H1 includes instruction content.

In addition, the instruction report H1 may include, for the target person 211, for example, exercise evaluation information G2 before the instruction report H1 was issued in the past, may include exercise evaluation information G2 after the instruction report H1 was issued in the past, or may include both of these.

The exercise information acquisition unit 114 acquires the exercise information G3 via, for example, the input unit 11, the communication unit 13, or the storage unit 14.

Here, for example, the exercise evaluation information acquisition unit 112 may generate and acquire the exercise evaluation information G2 based on the exercise information G3 acquired by the exercise information acquisition unit 114 and a predetermined evaluation rule.

The evaluation rule is a rule for evaluating the exercise information G3 and may include ideal exercise information, for example.

The instruction content acquisition unit 115 acquires information about the instruction content transmitted to the target person 211.

The exercise information evaluation unit 116 acquires exercise evaluation information about the target person 211 to whom the instruction content is transmitted.

Here, for example, the exercise information evaluation unit 116 may acquire such exercise evaluation information via the input unit 11, the communication unit 13, or the storage unit 14, or may acquire exercise information of the target person 211 via the input unit 11, the communication unit 13, or the storage unit 14 and acquire exercise evaluation information based on the exercise information.

In this embodiment, the instruction content acquisition unit 115 acquires content of instruction based on the target person information G1 including a plurality of parameters related to the target person 211 and the exercise evaluation information G2 about the evaluation of the exercise of the target person 211.

In addition, the exercise information evaluation unit 116 acquires the exercise evaluation information about the evaluation of the exercise of the target person 211 after the instruction.

When the content of instruction and the exercise evaluation information after the instruction are input, the learning model A1 during learning analyzes effective parameters among the plurality of parameters of the target person information G1.

Input and output of a trained model A2 will be described.

FIG. 2 is a diagram showing an example of input and output of the trained model A2 according to the embodiment.

The trained model A2 is a model in which the learning model A1 is in a learned state.

In this embodiment, the target person information G1 and the exercise evaluation information G2 are input to the trained model A2, and based on instruction content output from the trained model A2, the instruction report H1 including the instruction content is output.

Here, the trained model A2 does not necessarily need to output sentences, patterns, or the like of the instruction content as they are, and may output, for example, information that can identify the instruction content. As an example, a correspondence relationship between each of a plurality of pieces of identification information including numbers or the like and instruction content may be stored in a database or the like, the trained model A2 may output one or more pieces of identification information, and the instruction content corresponding to the identification information may be identified and included in the instruction report H1.

The target person information G1 will be described.

The target person information G1 input to the information processing device 1 will be described.

The target person information G1 is information about the target person 211 who receives the instruction of an exercise.

The target person 211 may input the target person information G1 via the information processing device 1, or an instructor who instructs the target person 211 may input the target person information G1, or a person concerned such as a guardian of the target person 211 may input the target person information G1.

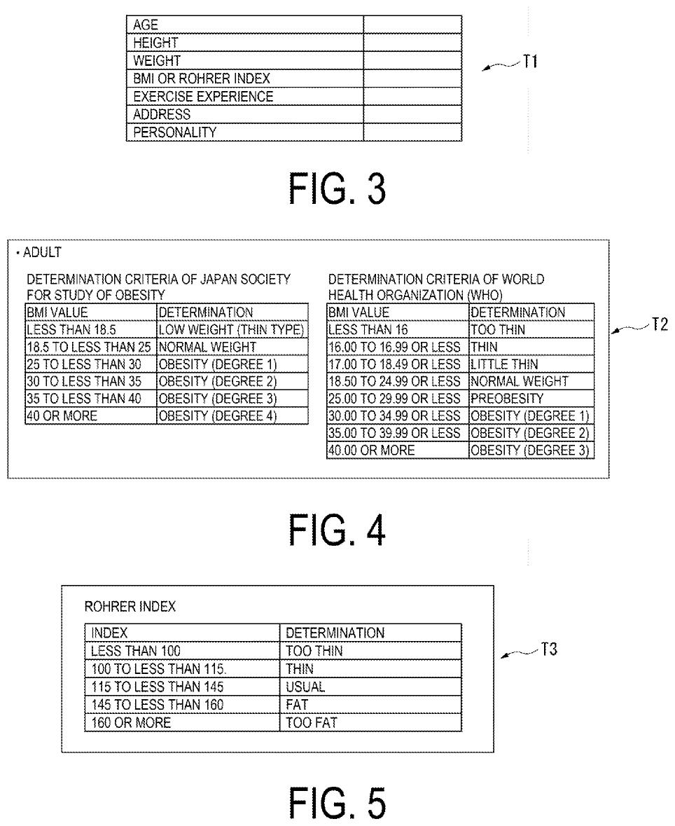

FIG. 3 is a diagram showing a first table T1 representing an example of the target person information G1 according to the embodiment.

The age is information indicating an age of the target person 211.

The height is information indicating a height of the target person 211.

The weight is information indicating a weight of the target person 211.

The BMI (Body Mass Index) is information about a BMI of the target person 211 who is 15 years old or older.

The Rohrer index is an index that can be substituted for the BMI when target person 211 is less than 15 years old.

FIG. 4 is a diagram showing a second table T2 representing an example of correspondence between a BMI value and a determination result according to the embodiment.

The BMI may be derived from a height and a weight, for example, instead of being input directly. In this case, for example, the BMI may be obtained by BMI=weight [kg]÷{height [m]}².

The BMI is called a body mass index and is a physique index indicating a degree of obesity calculated from a weight and a height.

When the age is less than 15 years old, the Rohrer index described below exists separately from the BMI, and when the age is 15 years old or older, the BMI is an international index.

FIG. 5 is a diagram showing a third table T3 representing an example of correspondence between a Rohrer index and a determination result according to the embodiment. The Rohrer index may be derived from a height and a weight instead of being input directly. In this case, for example, the Rohrer index may be obtained by, for example, Rohrer index=weight [kg]÷{height [m]}³×10.

The exercise experience is information indicating how long the exercise serving as a target of the instruction is performed, and may be represented by, for example, the number of years or months.

The address is information about an address of a place in which the target person 211 lives.

Here, it is possible to determine whether or not the target person 211 lives in a place far from the instructor from the information about the address. Based on this determination, it is possible to determine whether or not there are many opportunities to receive direct instruction from the instructor. For example, it may be determined that a person who lives far from the instructor has fewer opportunities to receive direct instruction from the instructor than a person who lives close to the instructor.

The personality is information about a personality of the target person 211.

As a mode for inputting the personality, a mode of answering from among several options may be used, or a descriptive mode may be used.

The options may be, for example, an “active personality”, a “never-give-up personality”, a “flexible personality”, a “tenacious personality” a “serious personality”, or a “calm personality”.

In this embodiment, among these options, the following three personalities will be exemplified.

Characteristics of the “never-give-up personality” include a characteristic of being resilient, that is, not giving up even if having failed, a characteristic of being persistent, that is, continuing until success, and a characteristic of being competitive, that is, being able to tackle things with a desire to improve;

Characteristics of the “flexible personality” include a characteristic of high adaptability, that is, being able to respond obediently to what is said, and a characteristic of adaptation to circumstances, that is, being able to respond appropriately in accordance with a situation at hand.

Characteristics of the “tenacious personality” include a characteristic of an inquisitive mind, that is, to deeply understand things and master them until one is satisfied with them, and a characteristic of not being swayed by others, that is, to follow through on whatever he or she decides to do until he or she is satisfied with it.

The exercise evaluation information G2 input to the information processing device 1 will be described.

The exercise evaluation information G2 is information indicating the evaluation of the exercise performed by the target person 211.

The exercise performed by the target person 211 includes various kinds of exercises such as soccer, baseball, tennis, dance, golf, and Judo.

The information about the exercise is acquired by providing a sensor that acquires a movement of the target person 211.

The sensor is a device that acquires, for example, acceleration information, positioning information of a global navigation satellite system (GNSS) such as a global positioning system (GPS), or the like.

In this embodiment, such a sensor is provided in each of the first detection device 251 and the second detection device 271.

For example, in an exercise that does not use a tool, such as dancing or judo, the first detection device 251 is attached to the body of the target person 211.

For example, in an exercise using a tool, such as soccer, baseball, tennis, or golf, one or both of the first detection device 251 attached to the body of the target person 211 and the second detection device 271 attached to the tool 231 used are used.

When the sensor is attached to the body, the sensor may be attached to, for example, the limbs, waist, or torso of the body.

In this embodiment, as an example of the exercise, a case in which the present disclosure is applied to soccer will be described, and an exercise of kicking a ball will be described.

An attachment position of the sensor on the target person 211 is, for example, a leg kicking a ball. In this embodiment, the attachment position of the sensor on the target person 211 is one leg, but the present disclosure is not limited thereto, and any one of both legs, both arms, the waist, and the torso may be set as the attachment position, or a plurality of positions obtained by combining both legs, both arms, the waist, and the torso may be set as the attachment position of the sensor

Also, when the attachment position of the sensor on the target person 211 is a leg, the attachment position may be a knee of the leg, for example.

A motion is a series of movements for kicking a ball.

Further, the movement may be called an action instead.

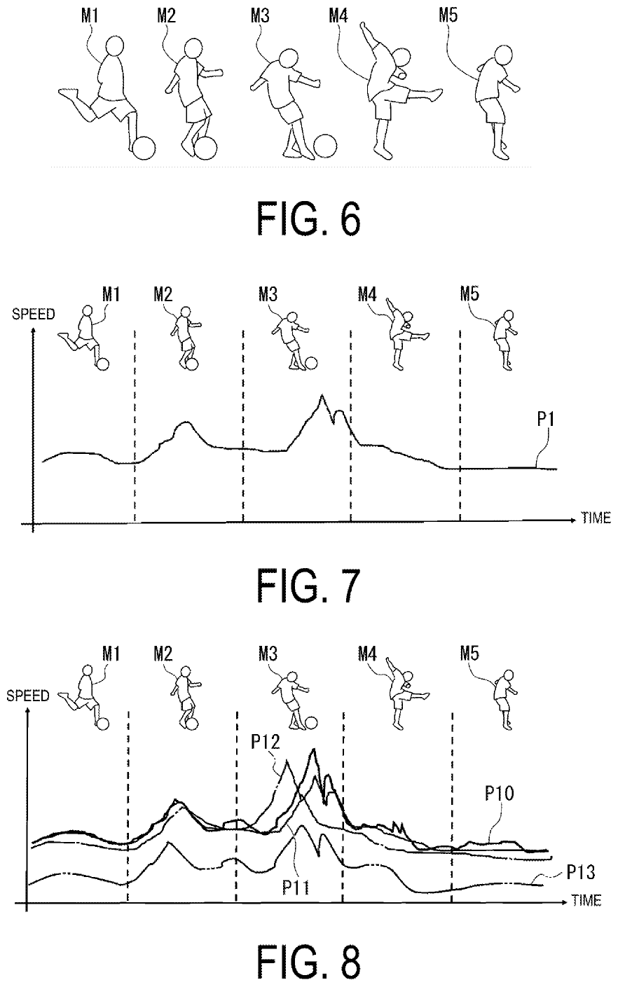

FIG. 6 is a diagram showing an example of the series of movements for kicking a ball according to the embodiment.

In the example of FIG. 6, five stage movement states are shown from a state before the target person 211 kicks the ball to a state after the target person 211 kicks the ball.

The five stage movements according to this embodiment will be described. A first movement M1, which is a movement at a first stage, is a movement from the start of running to swinging down of a leg of the target person 211. A second movement M2, which is a movement at a second stage, is a movement from the swinging down of the leg to immediately before kicking the ball. A third movement M3, which is a movement at a third stage, is a movement from immediately before kicking the ball to immediately after kicking the ball. A fourth movement M4, which is a movement at a fourth stage, is a movement from immediately after kicking the ball to swinging up the leg. A fifth movement M5, which is a movement at a fifth stage, is a movement from swinging up the leg to swinging down the leg. Here, “immediately before kicking the ball” refers to a state in which a distance between a foot kicking the ball and the ball is within a predetermined range, and “immediately after kicking the ball” refers to a state in which the distance between the foot kicking the ball and the ball is outside of the predetermined range. The predetermined range is not particularly limited, but is, for example, a range in which the distance between the foot kicking the ball and the ball is within 20 cm. Further, the five stage movements are not limited thereto.

The exercise information G3 is waveform data indicating a speed of the leg measured by the sensor attached to the leg kicking the ball. From the exercise information G3, information about whether or not an impact is given to the ball at the point of maximum leg speed is acquired. The information includes, for example, information about a speed at which the thigh, knee, leg, or the like is swung up.

In this embodiment, the exercise information G3 is the waveform data indicating the speed of the leg kicking the ball, but the present disclosure is not limited thereto and may be waveform data indicating the movement of the body when jumping, for example. In this case, the sensor measures information such as a speed of bending the leg at the time of a movement of sinking a body such as a leg, an angle of the back that sinks at the time of the movement, a speed of jumping at the time of a jumping movement, and an inclination of the body when jumping at the time of the movement.

In addition, the exercise information G3 may be waveform data indicating a movement of the body at the time of performing a step of moving left and right, up and down, or the like. In this case, the sensor measures information such as an inclination of an upper half body, a speed of the step, a rhythm of the step, or the like.

Further, the exercise information G3 may be waveform data indicating a movement of the body during running. In this case, the sensor measures information such as a ground contact time of a foot, a back and forth movement of the shoulder, a vertical movement of the head, a back and forth movement of the upper half body, and a horizontal movement of the upper half body.

Further, in this embodiment, an example of the exercise is soccer, but if the exercise for which the target person 211 is instructed is baseball, the waveform data measured by the sensor at the time of swinging a bat or the waveform data acquired by the sensor at the time of throwing a ball becomes the exercise information G3. If the exercise for which the target person 211 is instructed is golf, the waveform data measured by the sensor at the time of swinging a golf club is used as the exercise information G3.

FIG. 7 a diagram showing the exercise information G3 according to the embodiment and showing an example of a relationship between a time and a speed at the time of swinging up the leg or the like.

In the graph shown in FIG. 7, the horizontal axis represents time, the vertical axis represents speed, and the broken lines represent boundaries among the first movement M1 to the fifth movement M5, which are the five stage movements.

The graph shows a first relationship P1 between time and speed.

The exercise evaluation information G2 is, for example, information indicating evaluation of the exercise information G3 of the target person 211. The evaluation may be derived by, for example, comparing the exercise information G3 of the target person 211 with exercise information G3, which is ideal data regarding a coach, a professional athlete, or an athlete of the same generation with technical ability. The evaluation may show the derived data as a graph, a score, a comment, or the like.

FIG. 8 is a diagram showing the exercise information G3 including a plurality of patterns according to the embodiment and showing an example of a relationship between a time and a speed at the time of swinging up the leg or the like.

In the graph shown in FIG. 8, the horizontal axis represents time, the vertical axis represents speed, and the broken lines represent boundaries among the first movement M1 to the fifth movement M5, which are the five stage movements.

The graph shows the relationship between the time and the speed in the exercise information G3 and shows an ideal relationship P10 indicating an ideal relationship between the time and the speed, a 1a relationship P11 indicating a first example of the relationship between the time and the speed, a 2a relationship P12 indicating a second example of the relationship between the time and the speed, and a 3a relationship P13 indicating a third example of the relationship between the time and the speed.

In the case of data of the 1a relationship P11, for example, the exercise evaluation information G2 may be such that “the speed at the timing of kicking the ball is slower than in the ideal relationship P10 in the third movement M3”, and a comment such as “the speed at the timing of kicking the ball is slow” may be shown.

In the case of data of the 2a relationship P12, for example, the exercise evaluation information G2 may be such that “the timing of kicking the ball is earlier than in the ideal relationship P10 in the third movement M3”, and a comment such as “the timing of kicking the ball is early” may be shown.

In the case of date of the 3a relationship P13, as a comment, for example, the exercise evaluation information G2 may be such that “the speed is generally lower than in the ideal relationship P10 in the first movement M1 to the fifth movement M5”, and a comment such as “the speed is generally slow” may be shown.

The instruction report H1 includes an instruction that improves the evaluation of the exercise information G3 of the target person 211.

Here, any form may be used as the content described in the instruction report H1 including the instruction content or the like, or as a format of description.

For example, in the instruction report H1, one or more of sentences, figures, graphs, and the like may be described.

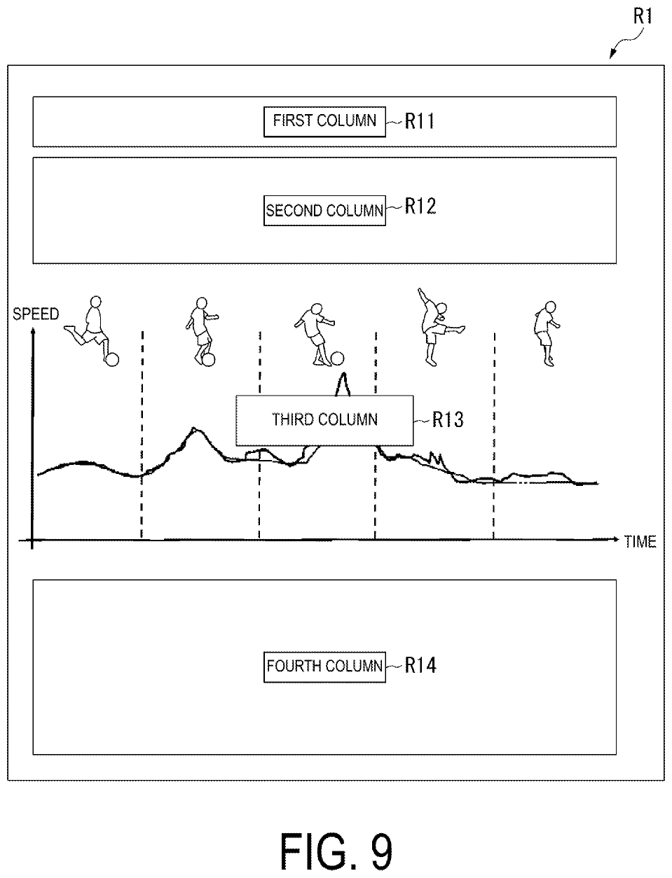

FIG. 9 is a diagram showing a schematic example of a first instruction report R1 according to the embodiment.

The first instruction report R1 is an example of the instruction report H1.

The first instruction report R1 includes a first column R11 in which information such as a name is described, a second column R12 in which information such as a comprehensive evaluation of the exercise information G3 of the target person 211 is described, a third column R13 in which information related to the exercise information G3 of the target person 211 is described, and a fourth column R14 in which information such as instruction content for the target person 211 is described.

In the first column R11, for example, one or more pieces of information among a name, an affiliation, a gender, an age, and the like are described. The affiliation may be, for example, a school name, a company name, a region name, or a club name to which the person belongs.

In addition, in the first instruction report R1, the date when the current exercise information G3 is measured may be described, and the date when the previous exercise information G3 is measured may be further described.

In the second column R12, for example, one or more pieces of information among a total score, a ranking, a sentence representing a comprehensive evaluation, a graph representing a comprehensive evaluation, and the like are described. For example, the total score indicates the exercise information G3 of the target person 211 that is expressed as a score when a score in the ideal exercise information G3 is set as an upper limit. The ranking is, for example, a result of comparing a total score of the target person 211 with a total score of another target person 211, which is shown as a rank.

In addition, in the second column R12, an evaluation obtained by comparing a measurement result of the current exercise information G3 of the target person 211 with a result of the previous exercise information G3 of the target person 211 may be shown.

In the third column R13, for example, one or more pieces of information among suggestions for the exercise, actually measured states of the exercise, a difference between the exercise information G3 of the target person 211 and the ideal exercise information G3, or a difference between the exercise information G3 of the target person 211 and average exercise information G3 based on the exercise information G3 of other target persons 211, and the like may be described.

In addition, in the third column R13, the first movement M1 to the fifth movement M5 may be described. As specific examples, one or two or more pieces of information may be described for the first movement M1 to the fifth movement M5, such as an angle of swinging up the leg in the first movement M1, a speed of swinging down the leg in the second movement M2, a timing at which the foot touches the ball in the third movement M3, an angle of swinging back the leg in the fourth movement M4, and a posture after kicking in the fifth movement M5. Also, one or more pieces of information among patterns respectively representing the first movement M1 to the fifth movement M5, predetermined waveform data measured through the plurality of movements, and the like may be described. As the waveform data, for example, the exercise evaluation information G2 of the target person 211 may be described.

Further, information such as a kick speed indicating a speed of the foot when kicking the ball, a kick impact force indicating a force applied to the ball at the time of kicking the ball may be described.

In the fourth column R14, for example, instruction content based on the exercise evaluation information G2 may be described. The instruction content may be advice for the entire exercise information G3, may be separate advice for each of the first movement M1 to the fifth movement M5, or may be advice for any one of the first movement M1 to the fifth movement M5.

Here, in the example of FIG. 9, the first instruction report R1 is divided into the first column R11 to the fourth column R14 and the information corresponding to each column is described, but the content of the instruction report H1 may be arbitrary, and it is sufficient that at least the information about the instruction content to the target person 211 is described. Also, the plurality of columns do not necessarily need to be clearly divided as in the example of FIG. 9.

For example, in the instruction report H1, the instruction content may be described in any location, or may be described in a plurality of locations.

Further, the instruction content may be expressed in any form and may be expressed using, for example, one or more of a sentence or a pattern representing the instruction content, a sentence or a pattern representing a target, a sentence or a pattern representing the difference between the exercise information G3 of the target person 211 and the ideal exercise information G3, and the like.

In this embodiment, the instruction content is instruction content for increasing the evaluation of the exercise information G3 of the target person 211. The evaluation may be expressed, for example, by a degree of similarity between the exercise information G3 of the target person 211 and the ideal exercise information G3.

The instruction may be an instruction for each of the first movement M1 to the fifth movement M5 obtained by dividing the exercise performed by the target person 211, or may be an instruction related to one of the divided first movement M1 to the fifth movement M5.

The instruction content is described in the instruction report H1 as different content based on the target person information G1.

The instruction content described in the instruction report H1 will be described.

The instruction content includes instruction related to a movement, and the instruction related to the movement is classified into simple content, typical content, and advanced content.

The simple content is, for example, content about how to use the body, and as a specific example, content such as “swing up the leg largely” or “bend the knee and kick the ball”.

The typical content is, for example, content about a point to be aware of, and as a specific example, content such as “awareness of a speed at the time of largely swinging up the leg” or “awareness of a force applied to the foot at the time of bending the knee and kicking the ball”.

The advanced content is, for example, content about a more detailed point to be aware of, and as a specific example, content such as “awareness of a speed below the knee at the time of swinging up the leg largely” or “awareness of a force applied to a big toe of the foot at the time of bending the knee and kicking the ball”.

The number of movements for which the instruction content is shown in the instruction report H1 will be described. The number of movements for which the instruction content is shown differs between the simple content and the advanced content.

The simple content indicates, for example, instruction content for one of the first movement M1 to the fifth movement M5.

The advanced content indicates, for example, instruction content for two or more movements among the first movement M1 to the fifth movement M5.

Instruction content about a practice method in the instruction report H1 will be described. The instruction about the practice method is divided into simple content, typical content, and advanced content.

The simple content is, for example, content about “doing the number of repetitions”.

The typical content is, for example, content about “doing the number of repetitions and performing light muscle training”. Examples of the muscle training include light muscle training such as standing on one leg, and muscle training such as abdominal muscle training or squats. Also, for muscle training such as abdominal muscle training or squats, instruction content to perform a reduced number of repetitions may be used.

The advanced content is, for example, content about “doing the number of repetitions and performing appropriate muscle training and core training”. Further, instruction content for increasing the number of repetitions of muscle training such as abdominal muscle training or squats may be used.

Proposals for the practice method in the instruction report H1 will be described. The proposals for the practice method differ in a simple communication method, a sophisticated communication method, and a communication method respecting the person's wishes.

The simple communication method is, for example, a communication method of suggesting the practice method using general terms.

The advanced communication method is, for example, a communication method for proposing a practice method while communicating technical terms or technical knowledge.

The communication method respecting the person's wishes is, for example, a communication method in which, in addition to a comment on the practice method already performed by the target person 211, the practice method is communicated as information rather than a proposal.

A specific example will be shown.

A specific example of a first target person will be described.

The specific example will be shown for a case in which the target person 211 is the first target person.

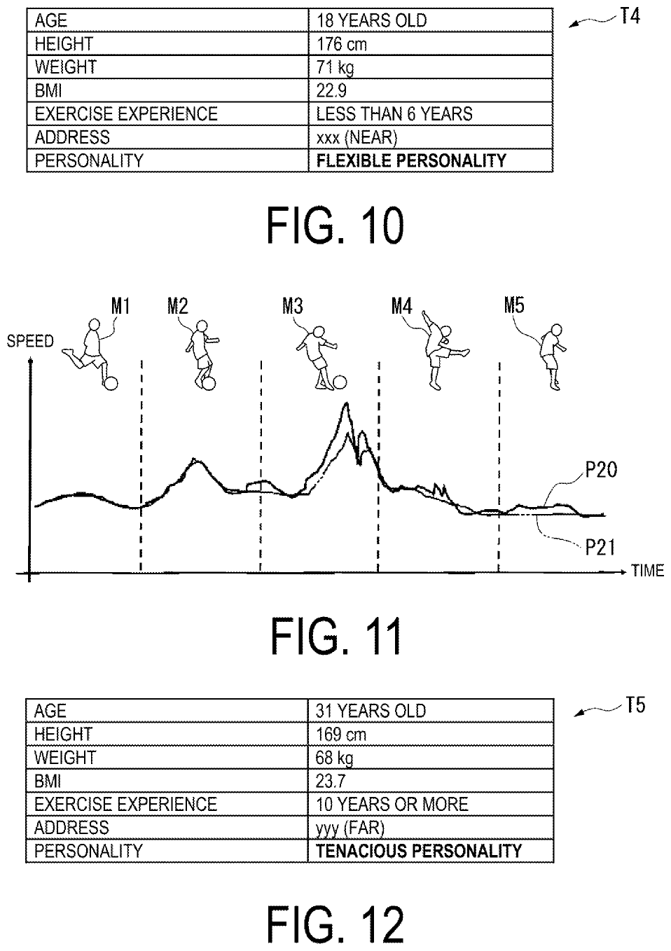

FIG. 10 is a diagram showing a fourth table T4 representing an example of first target person information according to the embodiment.

FIG. 11 is a diagram showing an example of the relationship between the time and the speed in the exercise information G3 of the first target person according to the embodiment.

In the graph shown in FIG. 11, the horizontal axis represents time, and the vertical axis represents speed. The graph shows a 1b relationship P21 between the time and the speed in the exercise information G3 of the first target person. Also, the graph shows a first ideal relationship P20 between the time and the speed in the exercise information G3 that is ideal for the exercise performed by the target person 211.

In the first target person information, which is the target person information G1 of the first target person, the age is 18 years old, the height is 176 cm, the weight is 71 kg, the BMI is 22.9, the exercise experience is less than 6 years, the address is “xxx”, which is an address of a place close to the instructor, and the personality is a flexible personality.

The exercise evaluation information G2 of the first target person is such that “the speed of the timing of kicking the ball is slower than in the first ideal relationship P20 in the third movement M3”, and a comment such as “the speed of the timing of kicking the ball is slow” is shown.

The instruction content to the first target person is instruction content such as “instruction about the movement of swinging down the leg and the movement of kicking up the leg in addition to the movement of kicking the ball is given and conveyed along with technical knowledge in addition to moderate muscle training”.

Instruction content of a first pattern, which is a pattern of the first target person, will be described.

In the machine learning according to this embodiment, the advanced content is adopted as the instruction content about the movement, and specifically, the instruction content is about a more detailed point to be aware of. The point is, for example, “awareness of the speed under the knee at the time of swinging up the leg largely” or “awareness of the force applied to the big toe of the foot at the time of bending the knee and kicking the ball”.

The reason for adopting such instruction content is that it can be determined from the age and years of experience of the first target person that he or she understands how to move the body, and he or she can practice efficiently by being aware of a detailed way of moving the body.

On the other hand, in the machine learning according to this embodiment, the simple content and the typical content are not adopted as the instruction content about the movement.

The simple content is instruction content about how to use the body, and is, for example, “swing up the leg largely”, “bend the knee and kick the ball”, or the like.

The typical content is instruction content about a point to be aware of, and is, for example, “awareness of the speed at the time of swinging up the leg largely” or “awareness of the force to the foot at the time of bending the knee and kicking the ball”.

The reason why such instruction content is not adopted is that it can be determined from the age and years of experience of the first target person that he or she can understand how to move the body, and thus even if he or she is informed of what has been understood, efficient practice cannot be achieved.

In the machine learning according to this embodiment, for the number of movements indicating the instruction content, the advanced content is adopted, and specifically, the instruction content is shown for two or more movements among the first movement M1 to the fifth movement M5.

The reason for adopting such instruction content is that the first target person is accustomed to the movement of kicking the ball due to his or her age and years of experience, and thus it is possible to make him or her aware of linked movements.

On the other hand, in the machine learning according to this embodiment, for the number of movements indicating the instruction, the simple content is not adopted, and specifically, the instruction content is shown for one of the first movement M1 to the fifth movement M5.

The reason why such instruction content is not adopted is that it is more effective to make him or her aware of the linked movements than to make him or her aware of movements separately.

In the machine learning according to this embodiment, the advanced content is adopted as the instruction content about the practice method, and specifically, the instruction content is not only about the number of repetitions but also about moderate muscle training.

The reason for adopting such instruction content is that it can be determined that a physique of the first target person is not before a growth period, and performing moderate muscle training will not inhibit the growth.

On the other hand, in the machine learning according to this embodiment, the simple content and the typical content are not adopted as the instruction content about the practice method.

The simple content is instruction content for doing the number of repetitions.

The typical content is instruction content not only for the number of repetitions but also for performing light muscle training.

The reason why such instruction content is not adopted is that it can be determined from the physique of the first target person that a load on the body is insufficient in the simple content and the typical content, and efficient training cannot be performed.

In the machine learning according to this embodiment, the advanced communication method is adopted as a proposal for the practice method, and specifically, the practice method is proposed while technical terms and technical knowledge are communicated.

The reason for adopting such a proposal is that, even if a method is proposed from this side, the method can be flexibly adopted because of the personality, and that it is more effective to incorporate technical knowledge rather than years of experience.

On the other hand, in the machine learning according to this embodiment, the simple communication method and the communication method respecting the person's wished are not adopted as proposals for the practice method.

The simple communication method is a communication method for proposing the practice method using general terms. The reason why such a proposal is not adopted is that it can be determined from years of experience of the first target person that he or she can understand even if the communication is performed without using general terms.

The communication method respecting the person's wishes is a communication method in which, in addition to a comment on the practice method already performed by the first target person, the practice method is communicated as information rather than a proposal. The reason why such a proposal is not adopted is that it is determined that the practice performed by the first target person himself or herself may be inefficient, and thus it is better to present the proposal from this side.

A specific example of a second target person will be shown.

The specific example will be shown for a case in which the target person 211 is the second target person.

FIG. 12 is a diagram showing a fifth table T5 representing an example of second target person information according to the embodiment.

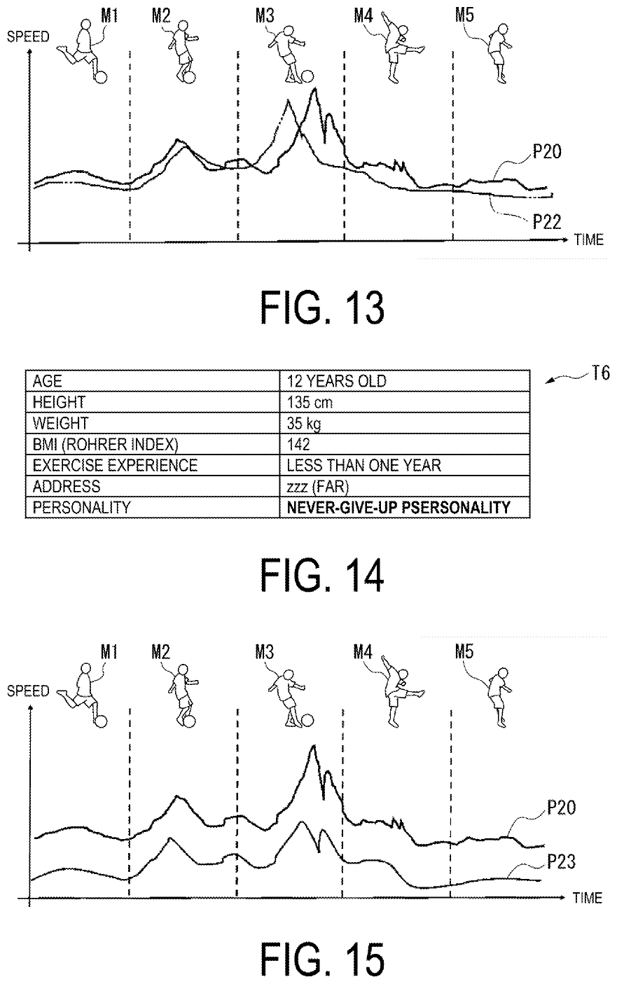

FIG. 13 is a diagram showing an example of the relationship between the time and the speed in the exercise information G3 of the second target person according to the embodiment.

In the graph shown in FIG. 13, the horizontal axis represents time, and the vertical axis represents speed.

The graph shows a 2b relationship P22 between the time and the speed in the exercise information G3 of the second target person. Also, the graph shows the first ideal relationship P20 between the time and the speed in the exercise information G3 that is ideal for the exercise performed by the target person 211.

In the second target person information, which is the target person information G1 of the second target person, the age is 31 years, the height is 169 cm, the weight is 68 kg, the BMI is 23.7, the exercise experience is 10 years or more, the address is “yyy”, which is an address of a place far from the instructor, and the personality is a tenacious personality.

The exercise evaluation information G2 of the second target person is such that “the timing of kicking the ball is earlier than in the first ideal relationship P20 in the second movement M2”, and a comment such as “the timing of kicking the ball is early” is shown.

The instruction content to the second target person is instruction content such as “instruction about the movement of swinging down the leg and the movement of kicking up the foot in addition to the movement of kicking the ball is given, and a practice method including moderate muscle training is communicated as information.

Instruction content of a second pattern, which is a pattern of the second target person, will be described.

In the machine learning according to this embodiment, the advanced content is adopted as the instruction content about the movement, and specifically, the instruction content is about a more detailed point to be aware of. The point is, for example, “awareness of the speed below the knee at the time of swinging up the leg largely” or “awareness of the force on the big toe of the foot at the time of bending the knee and kicking the ball”.

The reason for adopting such instruction content is that it can be determined from the age and years of experience of the second target person that he or she understands how to move the body, and he or she can efficiently practice by being aware of how to move the body finely.

On the other hand, in the machine learning according to this embodiment, the simple content and the typical content are not adopted as the instruction content about the movement.

The simple content is instruction content about how to use the body, and is, for example, “swing up the leg largely”, “bend the knee and kick the ball”, or the like.

The typical content is instruction content about a point to be aware of, and is, for example, “awareness of the speed at the time of swinging up the leg largely” or “awareness of the force to the foot at the time of bending the knee and kicking the ball”.

The reason why such instruction content is not adopted is that it can be determined from the age and years of experience of the second target person that he or she can understand how to move the body, and thus even if he or she is informed of what has been understood, efficient practice cannot be achieved.

In the machine learning according to this embodiment, for the number of movements indicating the instruction content, the advanced content is adopted, and specifically, the instruction content is shown for two or more movements among the first movement M1 to the fifth movement M5.

The reason for adopting such instruction content is that the second target person is accustomed to the movement of kicking the ball due to his or her age and years of experience, and thus it is possible to make him or her aware of linked movements.

On the other hand, in the machine learning according to this embodiment, for the number of movements indicating the instruction, the simple content is not adopted, and specifically, the instruction content is shown for one of the first movement M1 to the fifth movement M5.

The reason why such instruction content is not adopted is that it is more effective to make him or her aware of the linked movements than to make him or her aware of movements separately.

In the machine learning according to this embodiment, the advanced content is adopted as the instruction content about the practice method, and specifically, the instruction content is not only about the number of repetitions but also about moderate muscle training.

The reason for adopting such instruction content is that it can be determined from the age and the physique of the second target person that he or she is not before a growth period, and performing moderate muscle training will not inhibit the growth.

On the other hand, in the machine learning according to this embodiment, the simple content and the typical content are not adopted as the instruction content about the practice method.

The simple content is instruction content for doing the number of repetitions.

The typical content is instruction content not only for number of repetitions but also for performing light muscle training.

The reason why such instruction content is not adopted is that it can be determined from the age and the physique of the second target person that a load on the body is insufficient in the simple content and the typical content, and efficient training cannot be performed.

In the machine learning according to this embodiment, as a proposal for the practice method, the communication method respecting the person's wishes is adopted, and specifically, in addition to a comment on the practice method already performed by the second target person, the practice method is communicated not as a proposal but as information.

The reason for adopting such a proposal is that it can be determined that, since the personality of the second target person is a tenacious personality, he or she may repel and not perform the presented practice method when he or she is told to perform the presented practice method, and thus, there is a possibility that the second target person may understand and accept the practice method if a communication method of providing advice centered on the practice method already performed by the second target person and communicating the recommended practice method as information such as “This exercise is available” is used.

On the other hand, in the machine learning according to this embodiment, the simple communication method and the advanced communication method are not adopted as the proposal for the practice method.

The simple communication method is a communication method for proposing the practice method using general terms. The reason why such a proposal is not adopted is that he or she can understand even if the communication is performed without using general terms.

The advanced communication method is a communication method for proposing a practice method while communicating technical terms and technical knowledge. The reason why such a proposal is not adopted is that, due to the tenacious personality, there is a possibility that even if the practice method is proposed from this side, the practice method cannot be adopted.

A specific example of a third target person will be shown.

The specific example will be shown for a case in which the target person 211 is the third target person.

FIG. 14 is a diagram showing a sixth table T6 representing an example of third target person information according to the embodiment.

FIG. 15 is a diagram showing an example of the relationship between the time and the speed in the exercise information G3 of the third target person according to the embodiment.

In the graph shown in FIG. 13, the horizontal axis represents time, and the vertical axis represents speed.

The graph shows a 3b relationship P23 between the time and the speed in the exercise information G3 of the third target person. Also, the graph shows the first ideal relationship P20 between the time and the speed in the exercise information G3 that is ideal for the exercise performed by the target person 211.

In the third target person information, which is the target person information G1 of the third target person, the age is 12 years, the height is 135 cm, the weight is 35 kg, the Rohrer index instead of the BMI is 142, the exercise experience is less than one year, the address is “zzz”, which is an address of a place far from the instructor, and the personality is a never-give-up personality.

The exercise evaluation information G3 of the third target person is such that “the speed is generally slower than in the first ideal relationship P20 in the first movement M1 to the fifth movement M5”, and a comment such as “the speed is generally slow” is shown.

The instruction content to the third target person is instruction content such as “instruction about the movement of swinging up the foot is performed, and a practice method including light muscle training for increasing the speed of the movement of swinging up the foot is proposed”.

Instruction content of a third pattern, which is a pattern of the third target person, will be described. In the machine learning according to this embodiment, the simple content is adopted as the instruction content about the movement, and specifically, the instruction content is about how to use the body. The instruction content is, for example, “swing up the leg largely”, “bend the knee and kick the ball”, or the like.

The reason for adopting such instruction content is that it can be determined that the third target person is young in age and has low years of experience and may not be accustomed to how to move the body, and thus it is more effective to provide instruction on how to move the body itself, rather than instruction on detailed movements of the body.

On the other hand, in the machine learning according to this embodiment, the typical content and the advanced content are not adopted as the instruction content about the movement.

The typical content is instruction content about a point to be aware of, and is, for example, “awareness of the speed at the time of swinging up the leg largely” or “awareness of the force to the foot at the time of bending the knee and kicking the ball”.

The advanced content is instruction content about a more detailed point to be aware of, and is, for example, “awareness of the speed below the knee at the time of swinging up the leg largely” or “awareness of the force applied to the big toe of the foot at the time of bending the knee and kicking the ball”.

The reason why such instruction content is not adopted is that it can be determined from the third target person information of the third target person that, as he or she can be better aware of how to finely move the body, the age and years of experience are not so high.

In the machine learning according to this embodiment, the simple content is adopted for the number of movements indicating the instruction content, and specifically, the instruction content is set for one of the first movement M1 to the fifth movement M5.

The reason for adopting such instruction content is that it can be determined that the third target person is young in age and has low years of experience and has little experience of performing training while being aware of, and thus he or she is first accustomed to performing training while being aware of.

On the other hand, in the machine learning according to this embodiment, the advanced content is adopted for the number of movements indicating the instruction content, and specifically, the instruction content is shown for two or more movements among the first movement M1 to the fifth movement M5.

The reason why such instruction content is not adopted is that he or she may not be accustomed to performing training while being aware of, and thus it is determined that the movement may become dull by being aware of a plurality of movements.

In the machine learning according to this embodiment, the typical content is adopted as the instruction content about the practice method, and specifically, the instruction content is not only for the number of repetitions but also for performing light muscle training.

The reason for adopting such instruction content is that, in consideration that the personality of the third target person is a never-give-up personality, the motivation is less likely to be maintained only by doing the number of repetitions, and thus instruction to increase muscle strength through light muscle training or the like is performed to maintain the motivation.

On the other hand, in the machine learning according to this embodiment, the simple content and the advanced content are not adopted as the instruction content about the practice method.

The simple content is instruction content for doing the number of repetitions. The reason why such instruction content is not adopted is that it is determined from the personality of the third target person that it is difficult to maintain the motivation only with the simple content.

The advanced content is instruction content about not only the number of repetitions but also performing moderate muscle training.

The reason why such instruction content is not adopted is that the third target person is young in age and the physique is before a growth period, and thus it is determined that too much muscle training may hinder the growth of the third target person.

In the machine learning according to this embodiment, the simple communication method is adopted as a proposal for the practice method, and specifically, a practice method using general terms is proposed.

The reason for adopting such a proposal is that the third target person has little experience of being instructed in view of his or her age and living place far from the instructor, and thus he or she is first asked to practice with simple instruction.

On the other hand, in the machine learning according to this embodiment, as a proposal for the practice method, the advanced communication method and the communication method respecting the person's wishes are not adopted.

The advanced communication method is a communication method for proposing a practice method while communicating technical terms and technical knowledge. The reason why such a proposal is not adopted is that it is determined that the third target person may have little experience of being instructed in view of his or her age and living place, and thus he or she cannot firmly understand advanced instruction.

The communication method respecting the person's wishes is a communication method in which, in addition to a comment on the practice method already performed by the third target person, the practice method is communicated not as a proposal but as information. The reason why such a proposal is not adopted is that it is determined that the third target person may have little experience of being instructed in view of his or her age and living place, and thus he or she may not know what kind of practice to do.

Learning processing will be described.

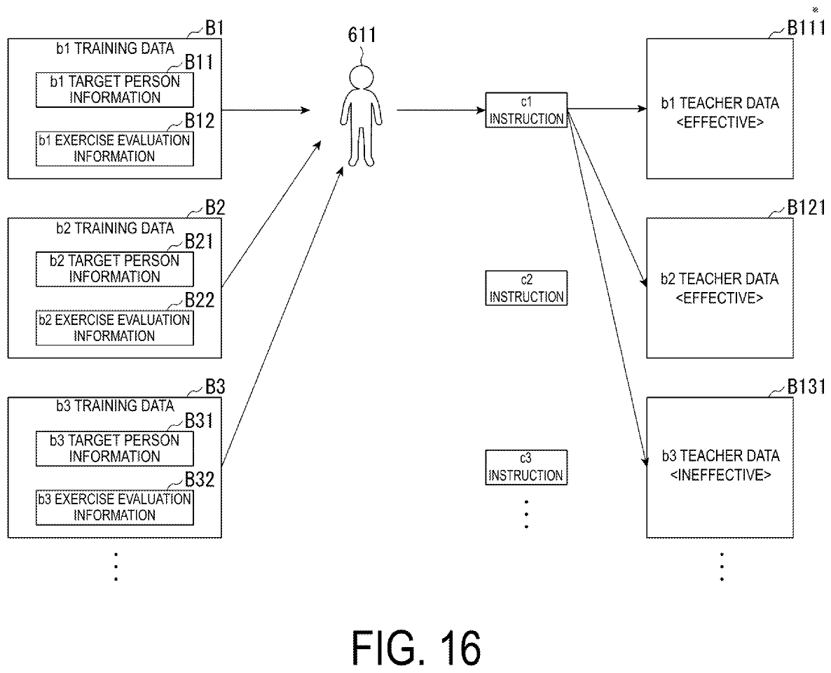

The learning processing will be described with reference to FIGS. 16 to 18.

FIG. 16 is a diagram schematically showing an example of generation of teacher data according to the embodiment. Also, training data may be called learning data or the like.

FIG. 16 shows b1 training data B1, b2 training data B2, and b3 training data B3.

The b1 training data B1 includes b1 target person information B11 and b1 exercise evaluation information B12. The b2 training data B2 includes b2 target person information B21 and b2 exercise evaluation information B22. The b3 training data B3 includes b3 target person information B31 and b3 exercise evaluation information B32.

Here, the b1 exercise evaluation information B12, the b2 exercise evaluation information B22, and the b3 exercise evaluation information B32 are exercise evaluation information having the same pattern B.

Also, the number of pieces of training data including exercise evaluation information having the same pattern may be, for example, four or more, or may be two, but in this embodiment, for convenience of description, a case in which the number is three will be described as an example. The number of pieces of such training data is actually, for example, a large number.

An instructor 611 may perform different kinds of instruction such as c1 instruction, c2 instruction, c3 instruction, and the like, and the c1 instruction will be described here as an example. In this embodiment, the c1 instruction is performed using an instruction report in which instruction content is described.

After the instructor 611 passes the instruction report including the content of the c1 instruction to a target person corresponding to the b1 training data B1, measurement of exercise is performed again for the target person on which the instruction has been reflected Then, the result of re-measurement after the target person receives the instruction report is used as b1 teacher data B111. The b1 teacher data B111 is, for example, exercise evaluation information obtained again for the target person and includes information about whether or not the instruction was effective.

For example, when the exercise evaluation information after the instruction is improved to such an extent that a predetermined condition is satisfied as compared with the exercise evaluation information before the instruction, the instruction is deemed effective, and in other cases, the instruction is deemed ineffective. The predetermined condition is, for example, that the similarity of the exercise information included in the exercise evaluation information with respect to the waveform data of the ideal exercise information is improved. In this embodiment, the case in which the instruction is effective is also referred to as “there is an effect”, and a case in which the instruction is ineffective is also referred to as “there is no effect”.

Here, the case in which the b1 teacher data B111 is generated from the b1 training data B1 and the c1 instruction has been described, but similarly, b2 teacher data B121 is generated from the b2 training data B2 and the c1 instruction, and b3 teacher data B131 is generated from the b3 training data B3 and the c1 instruction.

In the example of FIG. 16, the b1 teacher data B111 and the b2 teacher data B121 are when there is an effect, and the b3 teacher data B131 is when there is no effect.

Analysis in a model being trained A11 will be described.

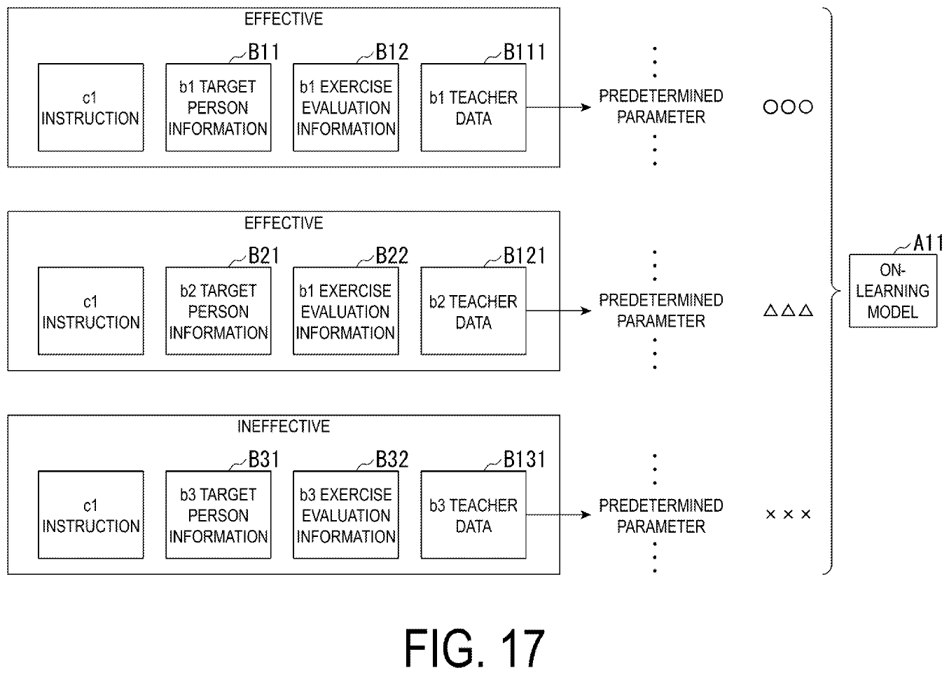

FIG. 17 is a diagram schematically showing an example of analysis in the model being trained A11 according to the embodiment.

Here, the model being trained A11 is a learning model in a state in which the learning model A1 is in a learning-in-progress stage. The learning model A1 changes from a state before learning to a trained model via the state of the model being trained A11.

From the results of the instruction, the model being trained A11 searches for a tendency as to the instruction is effective when which information is present in which parameter in the target person information.

In addition, the model being trained A11 may further determine a degree of effectiveness of the instruction. The degree of effectiveness may be represented by, for example, a numerical value representing the degree of effectiveness, and as an example, the numerical value increases as the effectiveness becomes greater.

In the example of FIG. 17, in the b1 target person information B11 of the target person corresponding to the b1 teacher data B111, the value of the predetermined parameter is “000”. As an example, there are two predetermined parameters, “a height of 176 cm” and “a weight of 71 kg”. In addition, in the b2 target person information B21 of the target person corresponding to the b2 teacher data B121, the value of the predetermined parameter is “AA”. As an example, there are two predetermined parameters, “a height of 169 cm” and “a weight of 68 kg”.

Further, in the b3 target person information B31 of the target person corresponding to the b3 teacher data B131, the value of the predetermined parameter is “XXX”. As an example, there are two predetermined parameters, “a height of 135 cm” and “a weight of 25 kg”.

The model being trained A11 analyzes the target person information to determine for which status of the target person the effect is exhibited and searches for the effective status.

In the example of FIG. 17, it is assumed that the BMI value of the target person corresponding to the b1 teacher data B111 and the BMI value of the target person corresponding to the b2 teacher data B121 indicates that the person has a solid body shape, but the BMI value of the target person corresponding to the b3 teacher data B131 does not indicate that.

Thus, the model being trained A11 learns that, when the exercise evaluation information is the pattern B, it is effective to give the c1 instruction to a target person having a solid body shape.

Also, in the example of FIG. 17, a case in which, for each target person, the target person information, the exercise evaluation information before the instruction, the instruction content, and the exercise evaluation information after the instruction are used in the learning processing has been shown, but for example, for the exercise evaluation information before the instruction and the exercise evaluation information after the instruction, the information may not necessarily be used as it is, and processed information may be used as long as the pattern of the exercise evaluation information before the instruction can be identified and the effect of the instruction can be identified.

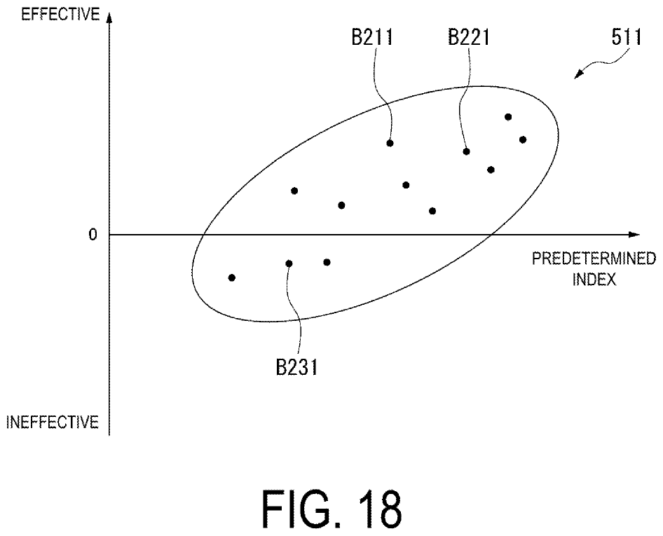

FIG. 18 is a diagram schematically showing an example of analysis content in the model being trained A11 according to the embodiment.

In the graph shown in FIG. 18, the horizontal axis represents a predetermined index, and the vertical axis represents a degree of effectiveness. On the vertical axis, there is no effect at a position of ±0, the effect increases upward in the positive range, and the reverse effect increases downward in the negative range.

FIG. 18 shows a distribution characteristic 511 according to analysis results assuming a case in which a large number of teacher data whose exercise evaluation information is the pattern B are analyzed. Also, the distribution characteristic 511 is an example for description and is not an actually obtained result.

In the example of FIG. 18, the predetermined index is, for example, a predetermined parameter itself or a value obtained from the predetermined parameter, and is a BMI value as an example

In the example of FIG. 18, a point corresponding to the b1 teacher data B111 corresponds to a b1 point B211, a point corresponding to the b2 teacher data B121 corresponds to a b2 point B221, and a point corresponding to the b3 teacher data B131 corresponds to a b3 point B231.

In this way, the learning model A1 learns correlations between the results of re-measurement after the target person 211 associated with the target person information G1 and the exercise evaluation information G2 receives the instruction report H1.

Also, in this embodiment, not only the information about whether or not there is an effect of the instruction but also the information about a degree of effectiveness of the instruction is used for the teacher data, but as another example, information about whether or not there is an effect of the instruction may be used for the teacher data, and information about a degree of effectiveness of the instruction may not be used.

As described above, the information processing device 1 according to this embodiment has the following configurations.

The target person information acquisition unit 111 acquires the target person information G1 about the target person 211.

The exercise evaluation information acquisition unit 112 acquires the exercise evaluation information G2 about the evaluation of the exercise of the target person 211. The storage unit 14 stores the trained model A2. When the target person information G1 and the exercise evaluation information G2 are input, the trained model A2 outputs the instruction content in which the evaluation of the exercise information G3 of the target person 211 is estimated to be improved.

The report output unit 113 inputs the target person information G1 and the exercise evaluation information G2 to the trained model A2 and outputs the instruction report H1 including the instruction content output from the trained model A2.

Accordingly, in the information processing device 1 according to this embodiment, it is possible to output the instruction report H1 suitable for each target person 211 and support the exercise of the target person 211.

For example, the information processing device 1 can infer, for each target person 211 receiving the exercise instruction, the instruction content that improves the evaluation of the exercise of the target person 211 among a plurality of instruction contents related to exercises, and output the instruction report H1 including the inferred instruction content, thereby supporting improvement of the exercise of the target person 211.

For example, in the information processing device 1 according to this embodiment, the exercise evaluation information G2 is information obtained by comparing target exercise information with the exercise information about the exercise of the target person 211.

Accordingly, the information processing device 1 according to this embodiment can support an exercise state of the target person 211 to approach a target exercise state.

Here, the target exercise information may be, for example, ideal exercise information.

Also, the target exercise information may be, for example, the same for all target persons 211, or may vary for each target person 211.

Further, an aspect in which the exercise is evaluated by a method different from comparison with the target exercise information may be used.

For example, in the information processing device 1 according to this embodiment, the target person information G1 includes information about the physique of the target person 211 and information about his or her age.

Accordingly, the information processing device 1 according to this embodiment can support the exercise based on the age and physique of the target person 211.

Also, an aspect in which one or both of the information about the physique of the target person 211 and the information about the age are not included in the target person information G1 may be used.

For example, in the information processing device 1 according to this embodiment, the target person information G1 includes information about the personality of the target person 211.

accordingly, the information processing device 1 according to this embodiment can support the exercise based on the personality of the target person 211.

Also, an aspect in which the information about the personality of the target person 211 is not included in the target person information G1 may be used.

For example, in the information processing device 1 according to this embodiment, the target person information G1 includes information about the place in which the target person 211 lives.

Accordingly, the information processing device 1 according to this embodiment can support the exercise based on the place in which the target person 211 lives.

Here, the living place may be identified by, for example, an address.

Also, an aspect in which the information about the place in which the target person 211 lives is not included in the target person information G1 may be used.

For example, in the information processing device 1 according to this embodiment, the storage unit 14 stores the exercise evaluation information G2 of the target person 211 in association with the target person information G1.

The exercise evaluation information acquisition unit 112 acquires the exercise evaluation information after the instruction report H1 is output from the report output unit 113.

The instruction report H1 includes the exercise evaluation information before the instruction report H1 is output from the report output unit 113 and the exercise evaluation information after the instruction report H1 is output from the report output unit 113.

Accordingly, in the information processing device 1 according to this embodiment, the exercise evaluation information before and after the output of the instruction report H1 can be presented to the target person 211.

For example, when the exercise evaluation information before the previous instruction report H1 is output to the target person 211 and the exercise evaluation information after the previous instruction report H1 is output to the target person 211 are included in the current instruction report H1, it is possible to understand how the evaluation of the exercise of the target person 211 is changed by the previous instruction report H1.

Also, an aspect in which one or both of the exercise evaluation information before outputting the instruction report H1 and the exercise evaluation information after outputting the instruction report H1 are not included in the instruction report H1 may be used.

For example, in the information processing device 1 according to this embodiment, the exercise evaluation information G2 includes the evaluation of the first movement in the exercise and the evaluation of the second movement in the exercise.

Accordingly, the information processing device 1 according to this embodiment can present evaluations for the plurality of movements in the exercise.

Here, in this embodiment, the first movement M1 to the fifth movement M5 are shown, but any two of these movements may be set as the first movement and the second movement.

Also, an aspect in which the evaluations for the plurality of movements in the exercise are not presented may be used.

For example, in the information processing device 1 according to this embodiment, the instruction report H1 includes a first instruction about the first movement and a second instruction about the second movement.

Accordingly, the information processing device 1 according to this embodiment can perform the instruction for the plurality of movements in the exercise.

Also, an aspect in which the instruction is not performed for the plurality of movements in the exercise may be used.

For example, in the information processing device 1 according to this embodiment, the instruction report H1 includes one of the first instruction about the first movement and the second instruction about the second movement.

Accordingly, in the information processing device 1 according to this embodiment, it is possible to give an instruction related to one of the plurality of movements in the exercise, and it is possible to easily understand the instruction content as compared with the case in which the instruction related to the plurality of movements is given.

Also, an aspect in which not only the instruction related to one of the plurality of movements in the exercise but also an instruction related to another movement is performed may be used.

For example, in the information processing device 1 according to this embodiment, the exercise information acquisition unit 114 acquires the exercise information G3 about the exercise of the target person 211 based on the information from the first detection device 251 attached to the target person 211.

Accordingly, the information processing device 1 according to this embodiment can acquire the exercise information G3 from the movement of the target person 211 itself.

Also, an aspect in which the function of the exercise information acquisition unit 114 that acquires the exercise information G3 based on the information from the first detection device 251 is not provided in the information processing device 1 may be used.

For example, in the information processing device 1 according to this embodiment, the first detection device 251 detects information about at least one of acceleration, position information, and angular velocity.

Accordingly, the information processing device 1 according to this embodiment can acquire the exercise information G3 based on one or more of acceleration, position information, and angular velocity from the movement of the target person 211 itself.

Also, an aspect in which exercise information based on information other than acceleration, position information, and angular velocity is acquired may be used.

For example, in the information processing device 1 according to this embodiment, the exercise information acquisition unit 114 acquires the exercise information G3 about the exercise of the target person 211 based on the information from the second detection device 271 attached to the tool 231 for the exercise.

Accordingly, the information processing device 1 according to this embodiment can acquire the exercise information G3 from the movement of the tool 231 used for the exercise.

Also, an aspect in which the function of the exercise information acquisition unit 114 that acquires the exercise information G3 based on the information from the second detection device 271 is not provided in the information processing device 1 may be used.

For example, in the information processing device 1 according to this embodiment, the second detection device 271 detects information about at least one of acceleration, position information, and angular velocity.

Accordingly, the information processing device 1 according to this embodiment can acquire the exercise information G3 based on one or more of acceleration, position information, and angular velocity from the movement of the tool 231 used for the exercise.

Also, an aspect in which movement information based on information other than acceleration, position information, and angular velocity is acquired may be used.

For example, in the information processing device 1 according to this embodiment, the trained model A2 is a learning model that analyzes the effective parameter among the plurality of parameters of the target person information G1 during learning when the instruction content based on the target person information G1 including the plurality of parameters related to the target person 211 and the exercise evaluation information G2, and the exercise evaluation information after the instruction are input.

Accordingly, in the information processing device 1 according to this embodiment, the learning model during learning can analyze the effective parameter among the plurality of parameters of the target person information G1 based on the input information.

Also, the present disclosure is not limited thereto, and any operation for learning may be performed as an internal operation of the learning model during learning.

For example, the information processing device 1 according to this embodiment has the following configurations.

The instruction content acquisition unit 115 acquires the instruction content based on the target person information G1 including the plurality of parameters for the target person 211 and the exercise evaluation information G2 about the evaluation of the exercise of the target person 211.

The exercise information evaluation unit 116 acquires the exercise evaluation information about the evaluation of the exercise of the target person 211 after the instruction.

The storage unit 14 stores the model being trained A11. When the instruction content and the exercise evaluation information after the instruction are input, the model being trained A11 analyzes the effective parameter among the plurality of parameters of the target person information G1.

Accordingly, in the information processing device 1 according to this embodiment, the model being trained A11 can analyze the effective parameter among the plurality of parameters of the target person information G1 based on the input information.

Also, in the information processing device 1 according to this embodiment, a case in which the target person 211 is a person who performs exercise such as soccer and provides support for the exercise has been described, but the present disclosure is not limited thereto and may be applied to a case in which the target person 211 performs another exercise.

A program for realizing the function of any constituent unit in any device described above may be recorded on a computer-readable recording medium, and the program may be read and executed by a computer system. The “computer system” as used here is assumed to include hardware such as an operating system (OS) or peripheral devices. The “computer-readable recording medium” is a storage device such as a portable medium such as a flexible disk, a magneto-optical disk, a read only memory (ROM), a compact disc (CD)-ROM, and a hard disk built into a computer system. The “computer-readable recording medium” is assumed to include a medium that holds a program for a certain period of time, such as a volatile memory provided inside of a computer system serving as a server or a client when the program is transmitted via a network such as the Internet or a communication line such as a telephone line. The volatile memory may be a RAM. The recording medium may be a non-transitory recording medium.

The above program may be transmitted from a computer system storing the program in a storage device or the like via a transmission medium or using transmission waves in a transmission medium to another computer system. The “transmission medium” that transmits the program refers to a medium having a function of transmitting information, such as a network such as the Internet or a communication line such as a telephone line.

The above program may be for realizing some of the above-described functions. The above program may be a so-called difference file, which can realize the above-described functions in combination with a program already recorded in the computer system. The difference file may be called a difference program.

The function of any constituent unit in any device described above may be realized by a processor. Each processing in the embodiment may be realized by a processor that operates based on information such as a program and a computer-readable recording medium that stores information such as a program. In the processor, the function of each unit may be realized by individual hardware, or the function of each unit may be realized by integrated hardware. The processor may include hardware, and the hardware may include at least one of a circuit for processing digital signals and a circuit for processing analog signals. The processor may be configured using one or both of one or more circuit devices or one or more circuit elements mounted on a circuit board. For the circuit device, an integrated circuit (IC) or the like may be used, and for the circuit element, a resistor, a capacitor, or the like may be used.

The processor may be a CPU. However, the processor is not limited to the CPU, and various processors such as a graphics processing unit (GPU) or a digital signal processor (DSP) may be used. The processor may be a hardware circuit using an application-specific integrated circuit (ASIC). The processor may be configured by a plurality of CPUs or may be configured by a hardware circuit including a plurality of ASICs. The processor may be configured by a combination of a plurality of CPUs and a hardware circuit including a plurality of ASICs. The processor may include one or more of an amplifier circuit, a filter circuit, and the like for processing analog signals.

Although the embodiment has been described in detail with reference to the drawings, specific configurations are not limited to this embodiment and include designs and the like without departing from the gist of the present disclosure.

### APPENDIX

<Configuration Example 1> to <Configuration Example 19> will be shown.

### Configuration Example 1

An information processing device including:
a target person information acquisition unit configured to acquire target person information about a target person;
an exercise evaluation information acquisition unit configured to acquire exercise evaluation information indicating evaluation of an exercise of the target person;
a storage unit configured to store a trained model that outputs instruction content in which, when the target person information and the exercise evaluation information are input, evaluation of exercise information of the target person is estimated to be improved; and
a report output unit configured to input the target person information and the exercise evaluation information to the trained model and output an instruction report including the instruction content output from the trained model.

### Configuration Example 2

The information processing device according to <Configuration Example 1>, wherein
the exercise evaluation information is information obtained by comparing target exercise information with exercise information about the exercise of the target person.

### Configuration Example 3

The information processing device according to <Configuration Example 1> or <Configuration Example 2>, wherein
the target person information includes information about a physique of the target person and information about an age thereof.

### Configuration Example 4

The information processing device according to any one of <Configuration Example 1> to <Configuration Example 3>, wherein
the target person information includes information about a personality of the target person.

### Configuration Example 5

The information processing device according to any one of <Configuration Example 1> to <Configuration Example 4>, wherein
the target person information includes information about a place in which the target person lives.

### Configuration Example 6

The information processing device according to any one of <Configuration Example 1> to <Configuration Example 5>, wherein
the storage unit stores the exercise evaluation information of the target person in association with the target person information,
the exercise evaluation information acquisition unit acquires the exercise evaluation information after the instruction report is output from the report output unit, and
the instruction report includes the exercise evaluation information before the instruction report is output from the report output unit and the exercise evaluation information after the instruction report is output from the report output unit.

### Configuration Example 7

The information processing device according to any one of <Configuration Example 1> to <Configuration Example 6>, wherein
the exercise evaluation information includes evaluation of a first movement in the exercise and evaluation of a second movement in the exercise.

### Configuration Example 8

The information processing device according to <Configuration Example 7>, wherein
the instruction report includes a first instruction for the first movement and a second instruction for the second movement.

### Configuration Example 9

The information processing device according to <Configuration Example 7>, wherein
the instruction report includes one of a first instruction for the first movement and a second instruction for the second movement.

### Configuration Example 10

The information processing device according to any one of <Configuration Example 1> to <Configuration Example 9>, further comprising an exercise information acquisition unit configured to acquire the exercise information about the exercise of the target person based on information from a first detection device attached to the target person.

### Configuration Example 11

The information processing device according to <Configuration Example 10>, wherein
the first detection device detects information about at least one of acceleration, position information, and angular velocity.

### Configuration Example 12

The information processing device according to any one of <Configuration Example 1> to <Configuration Example 9>, further comprising an exercise information acquisition unit configured to acquire the exercise information about the exercise of the target person based on information from a second detection device attached to the a tool for the exercise.

### Configuration Example 13

The information processing device according to <Configuration Example 12>, wherein
a second detection device detects information about at least one of acceleration, position information, and angular velocity.

### Configuration Example 14

The information processing device according to any one of <Configuration Example 1> to <Configuration Example 13>, wherein
the trained model is a learning model configured to analyze an effective parameter among a plurality of parameters of the target person information during learning when the instruction content based on the target person information including the plurality of parameters related to the target person and the exercise evaluation information and the exercise evaluation information after the instruction are input.

It is also possible to provide an information processing method performed by the information processing device described above.

### Configuration Example 15

An information processing method including:
acquiring target person information about a target person using a target person information acquisition unit of an information processing device;
acquiring exercise evaluation information indicating evaluation of an exercise of the target person using an exercise evaluation information acquisition unit of the information processing device;
storing, using a storage unit of the information processing device, a trained model that outputs instruction content in which, when the target person information and the exercise evaluation information are input, evaluation of exercise information of the target person is estimated to be improved; and
inputting the target person information and the exercise evaluation information to the trained model and outputting an instruction report including the instruction content output from the trained model using a report output unit of the information processing device.

It is also possible to provide a program recording medium that records a program executed by the processor in the information processing device as described above.

### Configuration Example 16

A program recording medium configured to record a program, the program causing a computer to execute:
a target person information acquisition function configured to acquire target person information about a target person;
an exercise evaluation information acquisition function configured to acquire exercise evaluation information indicating evaluation of an exercise of the target person; and
a report output function configured to input the target person information and the exercise evaluation information to a trained model stored in a storage unit configured to store the trained model that outputs instruction content in which, when the target person information and the exercise evaluation information are input, evaluation of exercise information of the target person is estimated to be improved, and output an instruction report including the instruction content output from the trained model.

### Configuration Example 17

An information processing device including:
an instruction content acquisition unit configured to acquire instruction content based on target person information including a plurality of parameters related to a target person and exercise evaluation information about evaluation of an exercise of the target person;
an exercise information evaluation unit configured to acquire the exercise evaluation information about the evaluation of the exercise of the target person after an instruction; and
a storage unit configured to store a model being trained in which, when the instruction content and the exercise evaluation information after the instruction are input, an effective parameter among the plurality of parameters of the target person information is analyzed.

It is also possible to provide a model being trained as described above.

### Configuration Example 18

A model being trained in which, when instruction content based on target person information including a plurality of parameters related to a target person and exercise evaluation information about evaluation of an exercise of the target person, and the exercise evaluation information after an instruction is input, an effective parameter among the plurality of parameters of the target person information is analyzed.

It is also possible to provide an information processing method performed by the information processing device as described above.

### Configuration Example 19

An information processing method including:
acquiring instruction content based on target person information including a plurality of parameters related to a target person and exercise evaluation information about evaluation of an exercise of the target person using an instruction content acquisition unit of an information processing device;
acquiring the exercise evaluation information about the evaluation of the exercise of the target person after an instruction using an exercise information evaluation unit of the information processing device; and
storing, using a storage unit of the information processing device, a model being trained in which, when the instruction content and the exercise evaluation information after the instruction are input, an effective parameter among the plurality of parameters of the target person information is analyzed.

1. An information processing device comprising:
a target person information acquisition unit configured to acquire target person information about a target person;
an exercise evaluation information acquisition unit configured to acquire exercise evaluation information indicating evaluation of an exercise of the target person;
a storage unit configured to store a trained model that outputs instruction content in which, when the target person information and the exercise evaluation information are input, evaluation of exercise information of the target person is estimated to be improved; and
a report output unit configured to input the target person information and the exercise evaluation information to the trained model and output an instruction report including the instruction content output from the trained model.

2. The information processing device according to claim 1, wherein
the exercise evaluation information is information obtained by comparing target exercise information with exercise information about the exercise of the target person.

3. The information processing device according to claim 1, wherein
the target person information includes information about a physique of the target person and information about an age thereof.

4. The information processing device according to claim 3, wherein
the target person information includes information about a personality of the target person.

5. The information processing device according to claim 3, wherein
the target person information includes information about a place in which the target person lives.

6. The information processing device according to claim 1, wherein
the storage unit stores the exercise evaluation information of the target person in association with the target person information,
the exercise evaluation information acquisition unit acquires the exercise evaluation information after the instruction report is output from the report output unit, and
the instruction report includes the exercise evaluation information before the instruction report is output from the report output unit and the exercise evaluation information after the instruction report is output from the report output unit.

7. The information processing device according to claim 1, wherein
the exercise evaluation information includes evaluation of a first movement in the exercise and evaluation of a second movement in the exercise.

8. The information processing device according to claim 7, wherein
the instruction report includes a first instruction for the first movement and a second instruction for the second movement.

9. The information processing device according to claim 7, wherein
the instruction report includes one of a first instruction for the first movement and a second instruction for the second movement.

10. The information processing device according to claim 1, further comprising an exercise information acquisition unit configured to acquire the exercise information about the exercise of the target person based on information from a first detection device attached to the target person.

11. The information processing device according to claim 10, wherein
the first detection device detects information about at least one of acceleration, position information, and angular velocity.

12. The information processing device according to claim 1, further comprising an exercise information acquisition unit configured to acquire the exercise information about the exercise of the target person based on information from a second detection device attached to a tool for the exercise.

13. The information processing device according to claim 12, wherein
the second detection device detects information about at least one of acceleration, position information, and angular velocity.

14. The information processing device according to claim 1, wherein
the trained model is a learning model configured to analyze an effective parameter among a plurality of parameters of the target person information during learning when the instruction content based on the target person information including the plurality of parameters related to the target person and the exercise evaluation information and the exercise evaluation information after the instruction are input.

15. An information processing method comprising:
acquiring target person information about a target person using a target person information acquisition unit of an information processing device;
acquiring exercise evaluation information indicating evaluation of an exercise of the target person using an exercise evaluation information acquisition unit of the information processing device;
storing, using a storage unit of the information processing device, a trained model that outputs instruction content in which, when the target person information and the exercise evaluation information are input, evaluation of exercise information of the target person is estimated to be improved; and
inputting the target person information and the exercise evaluation information to the trained model and outputting an instruction report including the instruction content output from the trained model using a report output unit of the information processing device.

16. (canceled)

17. (canceled)

18. A model being trained in which, when instruction content based on target person information including a plurality of parameters related to a target person and exercise evaluation information about evaluation of an exercise of the target person, and the exercise evaluation information after an instruction is input, an effective parameter among the plurality of parameters of the target person information is analyzed.

19. (canceled)
